(19) United States
(12) Patent Application Publication
(10) Pub. No.: US 2025/0318810 A1
(43) Pub. Date: Oct. 16, 2025

(54) METHOD AND SYSTEM FOR NONLINEAR FREQUENCY COMPOUNDING

(71) Applicant: SHENZHEN MINDRAY BIO-MEDICAL ELECTRONICS CO., LTD., Shenzhen (CN)

(72) Inventors: Yuling CHEN, San Jose, CA (US); Ching-Hua CHOU, Shenzhen (CN); Ting-Ian JI, San Jose, CA (US); Albert GEE, Shenzhen (CN)

(21) Appl. No.: 19/085,353

(22) Filed: Mar. 20, 2025

Related U.S. Application Data
(60) Provisional application No. 63/633,396, filed on Apr. 12, 2024.

(51) Int. Cl. A61B 8/00

(52) U.S. Cl. CPC A61B 8/5207; A61B 8/54

(57) ABSTRACT

An ultrasound imaging system is disclosed that performs enhanced B-mode imaging through a pulse inversion (PI) process. A controller transmits a PI sequence including a positive and a negative ultrasound pulse into biological tissue. A signal processing circuit receives echo signals resulting from this PI sequence and extracts three distinct signals: a direct current harmonic (DCH) signal, a fundamental signal at the transmitted frequency, and a second harmonic signal. Weights are assigned to these signals to create weighted signals spanning different nonlinear frequency bands. A final image is then generated from these weighted signals, improving image penetration, resolution, and clutter reduction compared to standard methods.

### CROSS-REFERENCE TO RELATED APPLICATION

The present application claims priority to U.S. Provisional Application No. 63/633,396, filed on Apr. 12, 2024, and titled “METHOD AND SYSTEM FOR NONLINEAR FREQUENCY COMPOUNDING.” The entire content of the above-identified application is incorporated herein by reference.

### FIELD OF THE INVENTION

The invention relates to ultrasound imaging, and more particularly, to a method and apparatus for improving and enhancing B-mode imaging, or Brightness mode imaging, by using nonlinear frequency compounding.

### BACKGROUND

B-mode imaging, or Brightness mode imaging, stands as a foundational technique within the field of medical ultrasound imaging. It generates two-dimensional cross-sectional images by interpreting the reflection (echo) intensity of ultrasound waves as they propagate through various tissues within the body. The brightness levels on the resulting images correspond to the echo intensities, providing critical information about the internal structures of the body. This technique is indispensable for diagnosing various conditions, guiding procedures, and monitoring fetal development among other applications.

Despite its widespread utility, B-mode imaging faces challenges, particularly related to image clarity and depth penetration. As acoustic waves travel through nonlinear tissues, they generate a spectrum of signals, including very low frequency signals centered at DC (0 Hz) and nonlinear 2^{nd} harmonic signals. Traditionally, the nonlinear 2^{nd} harmonic signals are used to improve the quality of the ultrasound images with less clutter and artifacts compared to those using just the fundamental frequency. On the other hand, the DC component is regarded as “noise” due to its very low frequency and is typically removed from the imaging process. This standard approach overlooks the potential utility of the DC signal, which may carry valuable information capable of enhancing image penetration, given its presence in the low frequency band. In addition, preliminary evaluations demonstrate that the DC signal is superior on clutter reduction and border enhancement compared to the regular fundamental signal and 2^{nd} harmonic signal.

In this disclosure, a novel system and method are described that capitalize on the regular pulse inversion transmission technique to not only generate the DC signal and the 2^{nd} harmonic signal but also the fundamental signal (and potentially other odd-order harmonic signals), simultaneously. By extracting these signals separately, the method allows for the creation of three, or more, distinct images based on each signal type. Furthermore, the proposed method and system employ selective compounding of the images derived from the DC component, 2^{nd} harmonic, and fundamental signals. This selective compounding provides flexibility to optimize image quality throughout varying tissue depths (among different patient groups, different body parts of a patient, different spots of a body part), thereby overcoming traditional limitations associated with signal extraction and image clarity.

Additionally, a key feature of this method and system is its ability to maintain frame rates on par with those achieved in traditional harmonic imaging using regular pulse inversion (PI). This ensures that the enhancements in image quality and depth penetration are achieved without sacrificing the speed and efficiency of the imaging process, marking a significant advancement in B-mode imaging technology.

### BRIEF DESCRIPTION OF THE DRAWINGS

FIG. 1 illustrates a schematic block diagram of an embodiment of an ultrasound imaging system implementing the present invention, according to one example embodiment.

FIG. 2 illustrates a mathematic model that indicates the source of the DC component signal, according to one example embodiment.

FIG. 3 illustrates a mathematic model that indicates how different signals are derived from combining the received signals the positive and negative transmit pulses, according to one example embodiment.

FIG. 4 illustrates a signal spectrum showing the DC component signal, the fundamental signal, and the 2^{nd} harmonic signal can be generated simultaneously with a regular PI transmit, according to one example embodiment.

FIG. 5 illustrates example images generated based on the DC component signal, the 2^{nd} harmonic signal, the fundamental signal, and the compounding image, according to one example embodiment.

FIG. 6 illustrates an example method for ultrasound imaging using nonlinear frequency compounding, according to one example embodiment.

FIG. 7 illustrates an example computing device in which any of the embodiments described herein may be implemented.

### DETAILED DESCRIPTION

Embodiments disclosed herein introduce an advanced approach to ultrasound imaging, primarily enhancing B-mode imaging by leveraging nonlinear frequency compounding and signal extraction. At the core of this technology is the simultaneous generation and utilization of multiple signal types: nonlinear harmonic signals, the fundamental signal, and a distinct DC component harmonic signal (DCH). This method diverges from traditional practices by not only recognizing the value of the DCH signal, traditionally deemed as noise, but also by allowing flexible weighted compounding the images generated based on these different non-linear signals. The resultant compounding images significantly improves image penetration and clarity. In addition, this method can be implemented using standard ultrasound imaging hardware, facilitating seamless integration into existing diagnostic frameworks.

In particular, this method enables the simultaneous generation of the DCH signal, the fundamental signal, and the second harmonic signal using a standard pulse inversion (PI) transmission sequence. This means that, without any need for additional transmit cycles or hardware modifications, all three signal types are extracted from the same transmit-receive event. This capability represents a core innovation: it not only leverages the full spectrum of nonlinear propagation signals, including those previously discarded, but also preserves system efficiency and compatibility with existing ultrasound platforms.

The potential applications of this technology are vast and include, but are not limited to, B-mode imaging for patients who are difficult to image due to depth or tissue composition challenges, enhanced needle-visualization imaging for medical procedures, trans-cranial imaging (TCI), where penetrating the skull with sufficient clarity has historically been problematic, and reduced clutter levels beneath fetal skull in obstetrics imaging.

Depending on the implementation, the enhanced B-mode imaging may include one or more of the following key features:

### DC Component Harmonic Signal (DCH)

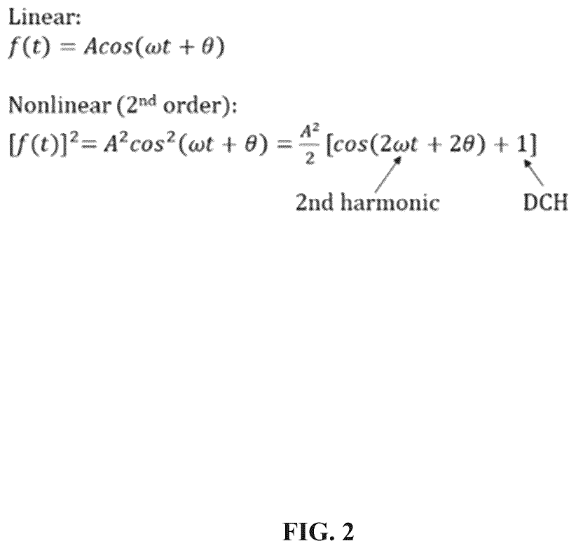

Tissue harmonic imaging (THI), and in particular 2^{nd} harmonic imaging, has gained widespread clinical adoption since the late 1990s, owing to its advantages such as reduced sidelobes. During the nonlinear propagation of acoustic waves, the generation of the 2^{nd} harmonic signal is invariably accompanied by a very low frequency signal centered at DC, which exhibits the same nonlinear characteristics as the 2^{nd} harmonic signal. FIG. 2 illustrates a mathematic model that indicates the source of the DC component signal. In the following description, the DC component signal is used as a special harmonic signal, denoted as DC harmonic (DCH) signal.

Historically, this DC harmonic (DCH) signal was largely dismissed as “noise” and did not receive the attention it deserved. However, with the advent of wide-band transducers capable of encompassing the low frequency range of the DCH, alongside sophisticated signal processing techniques that can individually extract signals across different spectral bands, the DCH signal now can be “extracted” and leveraged to enhance image quality, notably in terms of penetration depth, as well as clutter reduction and border enhancement.

### Spectral Band Extraction

This section describes how the DCH, fundamental signals, and the higher-order harmonic signals are extracted, by piggybacking on existing pulse inversion (PI) technique without using new hardware. As used herein, the term “DCH signal” refers to the very low-frequency component centered around 0 Hz that arises during the nonlinear propagation of ultrasound waves through tissue. The DCH signal shares nonlinear characteristics with even-order harmonic signals and has historically been treated as noise. The term “fundamental signal” refers to the component of the received echo corresponding to the original transmitted frequency of the ultrasound wave (e.g., the PI sequence), also known as the fundamental frequency. The term “higher-order harmonic signals” refers to nonlinear components of the echo signal occurring at integer multiples of the fundamental frequency—such as the second harmonic (2 f), third harmonic (3 f), and beyond.

The PI technique is a critical method widely used in Tissue Harmonic Imaging (THI) primarily because it enhances the suppression of unwanted fundamental frequency leakage into the harmonic signal. In THI, the goal is to create images using the harmonics generated by the ultrasound wave as it travels through the body, rather than the fundamental frequency (the original frequency of the ultrasound wave) because harmonic signals provide clearer images with less noise.

The PI technique operates by emitting a pair of ultrasound pulses into the tissue: one pulse is the positive (original) version of the waveform, and the second pulse is its negative (inverted) counterpart. The key to the PI technique lies in how the signals received back from these pulses are processed.

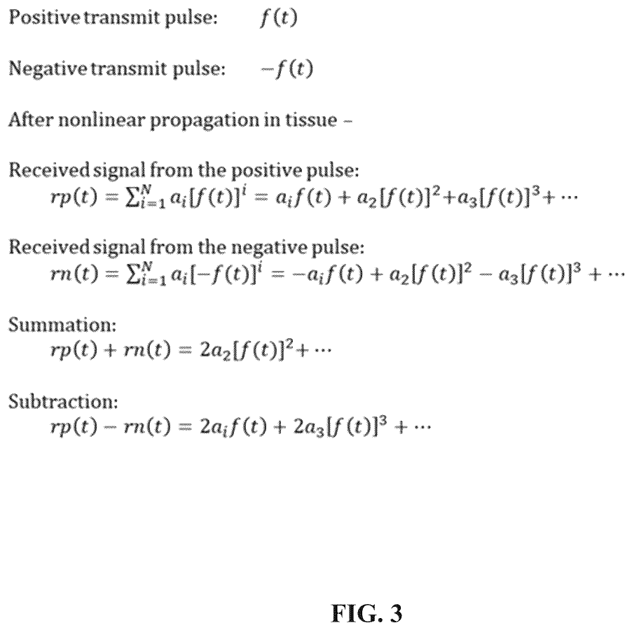

The mathematic model in FIG. 3 indicates how different signals are derived from combining the received signals from the positive and negative transmit pulses in PI. According to the model, once the positive and negative pulses have traversed through tissue, the signals from the positive pulse retain positive values, while those from the negative pulse display negative values at odd-numbered positions (with the first term representing the fundamental frequency component) and positive values at even-numbered positions. As a result, by adding the signals received from both the positive and negative transmit pulses, the system effectively cancels out the fundamental frequency component (i.e., aᵢf(t)−aᵢf(t)) and isolates the Direct Current Harmonic (DCH) signal (not shown in the model in FIG. 3) along with even-order harmonics (e.g., the 2^{nd} harmonic signals a₂[f(t)]²). This summation process, traditionally employed in imaging systems, aims to isolate and subsequently eliminate the DCH, focusing instead on extracting the 2^{nd} harmonic signals to improve image clarity and resolution.

In this disclosure, the novel system piggybacks on the PI transmission to isolate fundamental frequency components by subtracting the signals that arise from the transmission of both positive and negative pulses. As shown in FIG. 3, this subtraction process yields odd-order harmonics, including the fundamental (1st harmonic) and 3rd harmonic signals, etc. Therefore, by adopting the subtraction step as part of the standard PI transmission strategy, it is possible to simultaneously extract at least the DCH, the fundamental signal, and the 2^{nd} harmonic signal (assuming that the transducer's bandwidth does not accommodate higher-order harmonics). In certain configurations where the transducer's frequency range is sufficiently wide, it may be feasible to capture higher-order harmonic signals and incorporate them into a weighted compounding process to further refine image quality. This simultaneous extraction from a single PI transmission cycle is a key advantage of the system, allowing it to capture richer spectral content without any increase in acquisition time or hardware complexity.

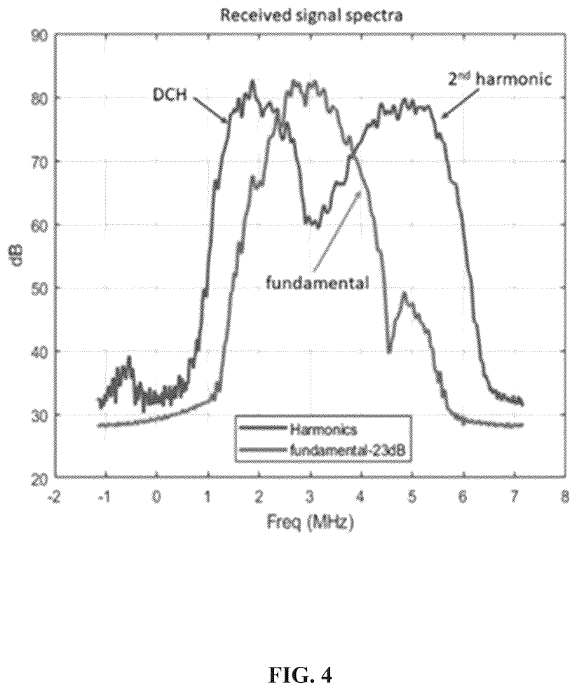

An example signal spectra is illustrated in FIG. 4 to show that the DCH component signal, the fundamental signal, and the 2^{nd} harmonic signal generated simultaneously with a regular PI transmit. As shown, the DCH has higher decibels (dB) in the lower frequency band, the fundamental signal has higher dB in the mid-level frequency band, and the 2^{nd} harmonic signal has higher dB in the higher frequency band. Essentially, the DCH signal is stronger in lower frequencies, the fundamental signal is stronger in mid-range frequencies, and the 2^{nd} harmonic signal exhibits greater strength in higher frequencies.

A person skilled in the art would appreciate that stronger signals might improve the clarity of the images by providing clearer differentiation between tissues or between tissues and fluid-filled spaces. This is particularly important for detecting fine details and structures within the body. In addition, lower-frequency signals (like those from the DCH) can penetrate deeper into the body because they are less attenuated than higher-frequency signals. Therefore, a stronger signal (higher dB) at these lower frequencies can potentially improve the visibility of deeper structures. Additionally, preliminary clinical evaluations show that the DCH signal helps clear out the “haze” clutter, which often appears in the near field of the image, better than fundamental or 2^{nd} harmonic signals, thus revealing more structures previously hidden by the “haze” Moreover, the DCH signal has stronger reflection on borders. Therefore, its potential in special applications, such as needle visualization, is large.

On the other hand, higher-frequency signals, such as the 2^{nd} harmonic, although they may not penetrate as deeply, can produce images with higher resolution because they can differentiate smaller structures. A higher dB in these signals can enhance the resolution further, making the images more detailed and easier to interpret.

Given that the unique characteristics of these signals render them more suitable for different applications in ultrasound imaging, the novel system described in this disclosure further introduces a post-detection weighted compounding method that allows users or researchers to assign different weights to these signals to generate the optimized images based on specific needs.

Post-Detection Compounding with Proper Weighting Settings (Weighted Compounding or Nonlinear Frequency Compounding)

Following the extraction of the Direct Current Harmonic (DCH), the fundamental signal, and the 2^{nd} harmonic signal from the Pulse Inversion (PI) transmission, a method of weighted compounding can be utilized. This method involves assigning specific depth-dependent weights to either the signals themselves or the images derived from these signals, effectively controlling their individual contributions to the final image composition. The “weights” in this context refer to the relative importance or influence assigned to each of these signals during the compounding process. By adjusting these weights, the imaging system can emphasize or de-emphasize certain aspects of the signals for different imaging depths based on the specific diagnostic needs or imaging objectives.

For instance, increasing the weight of the 2^{nd} harmonic signal, known for its higher resolution and clearer image quality, can enhance the overall clarity and detail in the compounded image. This is particularly useful for identifying fine structures or subtle pathologies.

If deeper tissue penetration is required, the system might assign a higher weight to the DCH signal or the fundamental signal, which are better at penetrating deeper into the body due to their lower frequencies and capturing features from deeper tissue Since the weights are depth-dependent, for shallower locations, the system can still weight the 2^{nd} harmonic signal more to maintain the high resolution. This adjustment allows the compounded image to achieve optimized image throughout the whole image.

The fundamental signal, which has a mid-level frequency, can provide a balanced view that includes both good penetration and reasonable resolution. Adjusting its weight can help in achieving the desired balance between contrast and specificity in the image, making it easier to differentiate between various tissue types. Moreover, since the fundamental signal occupies a separate spectral band and presents different characteristics than the harmonic signals, blending in the fundamental signal might further reduce the clutter levels.

By carefully selecting the weights for each signal, the non-linear compounding process can also help in minimizing artifacts and noise in the final image. For example, if certain signals are prone to producing artifacts under specific conditions, their weights can be reduced to diminish their impact on the overall image quality.

Different diagnostic scenarios may require focusing on different tissue characteristics. For instance, imaging vascular structures might benefit from a different weighting strategy than imaging solid organs or detecting tumors. The non-linear weighted compounding method allows for such customization, making it possible to tailor the imaging process to the precise needs of each examination.

In addition to depth-based weighting, the system may also employ case-dependent or application-specific weighting profiles, allowing it to optimize image quality based on the particular clinical scenario. For example, a preset weighting profile can be used to emphasize the DCH signal for applications that require better deep tissue penetration or clutter reduction, such as fetal skull imaging or transcranial Doppler. Conversely, scenarios like needle visualization may benefit from a higher weighting of the high-frequency 2^{nd} harmonic signal to improve edge definition and resolution.

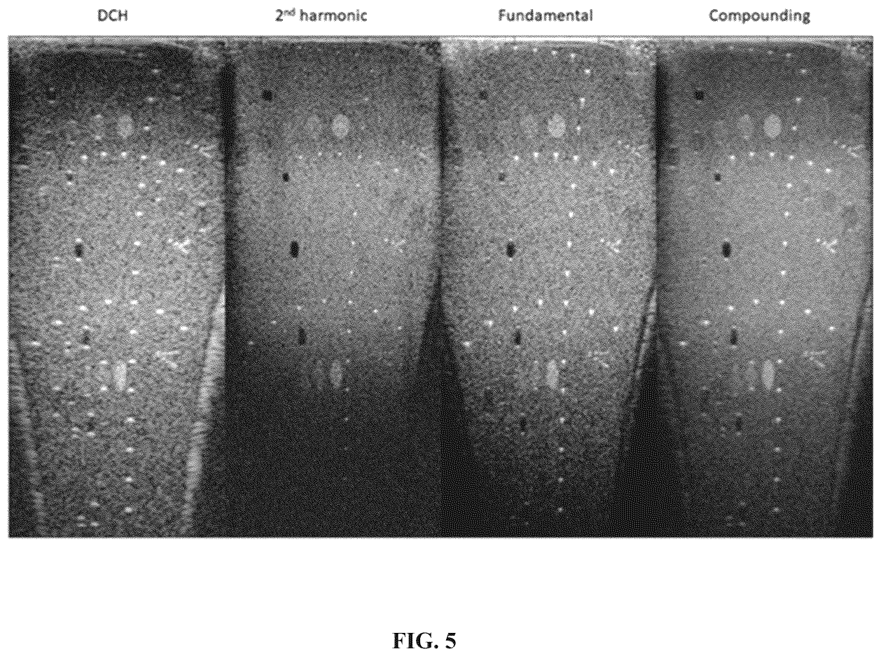

FIG. 5 illustrates example images generated based on the DCH signal, the 2^{nd} harmonic signal, the fundamental signal, and the compounding image. As shown, through properly designed weighting settings, the compounded image in FIG. 5 is optimized across various parameters. These include penetration depth, spatial resolution, contrast resolution, and the reduction of clutter, among others.

This process is also referred to as nonlinear frequency compounding because the weighted signals are derived from different frequency components—e.g., the DCH signal occupies the low-frequency band centered around 0 Hz, the fundamental signal corresponds to the original transmitted frequency of the ultrasound pulses, and the second harmonic signal lies in a higher-frequency band—each contributing distinct imaging characteristics to the final compounded image.

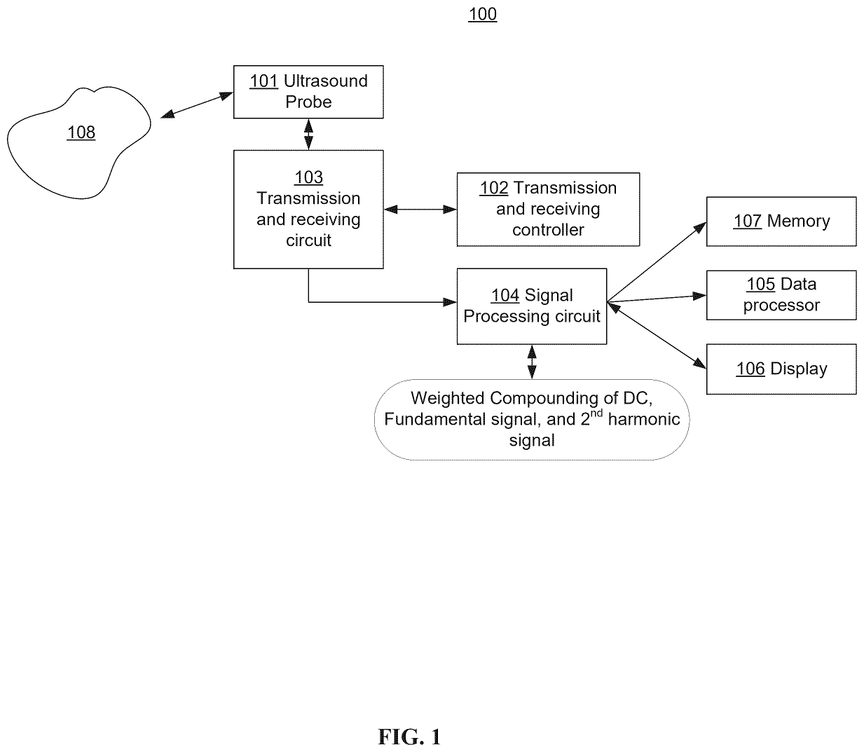

Turning the attention to FIG. 1, the illustrated apparatus 100 is an embodiment of the above-described ultrasound imaging system implementing the hybrid signal extraction (e.g., extracting DCH, the fundamental signal, and the 2^{nd} harmonic signal) followed by weighted compounding image generation. It should be noted that the components shown in FIG. 1 serve merely as examples. The actual composition of device 100 could vary, incorporating additional, fewer, or different components based on how it is implemented.

In some embodiments, the structure of an ultrasound apparatus 100 includes an ultrasound probe 101, a transmission and receiving controller 102, a data processor 105, a display device 106 and a memory 107. In a specific embodiment, the apparatus 100 further comprises a transmission and receiving circuit 103 and a signal processing circuit 104. The transmission and receiving controller 102 is in a signal connection with the ultrasound probe 101 by means of the transmission and receiving circuit 103, the ultrasound probe 101 is in a signal connection with the signal processing circuit 104 by means of the transmission and receiving circuit 103, an output end of the signal processing circuit 104 is connected to the data processor 105, and an output end of the data processor 105 is connected to the display device 106. The memory 107 is connected to the data processor 105.

The ultrasound probe 101 comprises a plurality of transducers which are also referred to as array elements, and the plurality of transducers are used to implement the mutual conversion of an electric pulse signal and ultrasound waves so as to transmit ultrasound waves to a biological tissue (e.g., a biological tissue in a human or animal body) 108 to be detected and receive ultrasound echoes reflected by the biological tissue. The plurality of transducers can be arranged in a row to form a linear array or arranged in a two-dimensional matrix to form an area array, and the plurality of transducers can also form a convex array. The transducers can transmit ultrasound waves excited by electric signals, or transform the received ultrasound echoes into electric signals. Therefore, each of the transducers can be either used to transmit ultrasound waves to a region of interest of a biological tissue, or used to receive ultrasound echoes reflected from the region of interest of the biological tissue.

When ultrasound detection is performed, a transmission sequence and a receiving sequence can control which transducers are used to transmit ultrasound and which transducers are used to receive ultrasound, or a transmission sequence and a receiving sequence can control the transducer to be used to transmit ultrasound waves or receive ultrasound echoes in a time slotted manner. All the transducers participating in ultrasound transmission can be simultaneously excited by the electric signal so as to simultaneously transmit ultrasound waves; or the transducers participating in ultrasound transmission can also be excited by several electric signals with a certain time interval, so as to continuously transmit ultrasound waves with a certain time interval.

The transmission and receiving controller 102 is used to generate a transmission/receiving sequence and output the transmission/receiving sequence to the ultrasound probe. The transmission sequence is used to control some or all of a plurality of array elements to transmit ultrasound waves to a region of interest of a biological tissue. The transmission sequence also provides transmission parameters (e.g., the amplitude, frequency, number of transmission, angle of transmission, mode and/or focused location, etc. of ultrasound waves). According to different purposes, the mode, transmission direction and focused location of the transmitted ultrasound can be controlled by means of adjusting the transmission parameters. The species of ultrasound waves may be pulse ultrasound waves, plane ultrasound waves, etc. The receiving sequence is used to control some or all of the plurality of array elements to receive ultrasound echoes reflected from the region of interest of the biological tissue.

The transmission and receiving circuit 103 is connected among the ultrasound probe and the transmission and receiving controller 102 and the signal processing circuit 104, and is used to transfer the transmission/receiving sequence controlled by the transmission and receiving controller 102 to the ultrasound probe 101 and transfer ultrasound echo signal received by the ultrasound probe 101 to the signal processing circuit 104.

In some embodiments, the PI transmission described in FIGS. 3-5 is implemented by the transmission and receiving controller 102 and carried out by the transmission and receiving circuit 103, in which positive signal transmission/receiving sequence and negative signal transmission/receiving sequence are performed sequentially (e.g., performing the positive sequence first, followed by the negative sequence).

The signal processing circuit 104 is used to process the ultrasound echo signals, for example, to perform filtering, amplification, beamforming and other processing for the ultrasound echo signal, so as to obtain ultrasound echo data. In a specific embodiment, the signal processing circuit 104 can be used to output the ultrasound echo data to the data processor 105, and can also firstly store the ultrasound echo data in the memory 107, such that when it is necessary to perform operation on the basis of the ultrasound echo data, the data processor 105 can read the ultrasound echo data from the memory 107. The memory 107 is used to store data and programs. The programs include a system program of the ultrasound apparatus, various application programs, or algorithms for realizing various specific functions. The data processor 105 is used to acquire the ultrasound echo data after the ultrasound echo being processed, and generate an ultrasound image according to the processed ultrasound echo data.

In some embodiments, the hybrid signal extraction process described in FIGS. 3-5 is implemented by the signal processing circuit 104, which performs the even-order harmonic signal extractions (e.g., to extract DCH and the 2^{nd} harmonic signal) as well as odd-order harmonic signal extractions (e.g., to extract the fundamental signal).

In some embodiments, the weighted compounding process described in FIGS. 4-5 may also be implemented in the signal processing circuit 104. For instance, the weights assigned to the different signals may be adjusted and transmitted to the signal processing circuit 104 to generate the weight-compounded image based on the signals and the weights.

The display device 106 may also be used to display detection results, for example, ultrasound images, calculation results, graphic charts or text description.

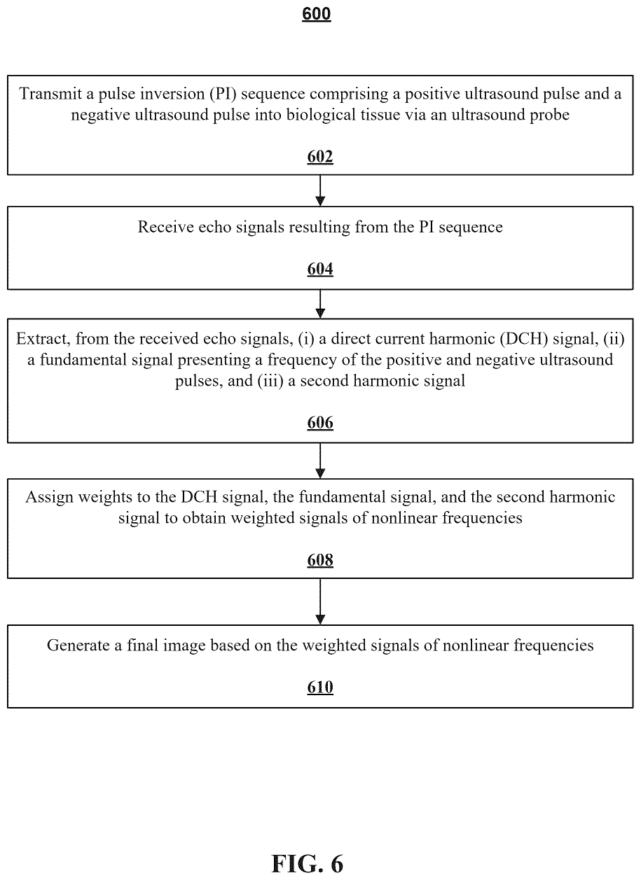

FIG. 6 illustrates an example method for ultrasound imaging using nonlinear frequency compounding, according to one example embodiment. In some implementations, one or more process blocks of FIG. 6 may be performed by a device.

As shown in FIG. 6, process 600 may include transmitting a pulse inversion (PI) sequence having a positive ultrasound pulse and a negative ultrasound pulse into biological tissue via an ultrasound probe (block 602). For example, the device may transmit a PI sequence as described above. Also, as shown in FIG. 6, process 600 may include receiving echo signals resulting from the PI sequence (block 604). For example, the device may receive echo signals resulting from the PI sequence as described above. Further, as shown in FIG. 6, process 600 may include extracting from the received echo signals (i) a direct current harmonic (DCH) signal, (ii) a fundamental signal presenting a frequency of the positive and negative ultrasound pulses, and (iii) a second harmonic signal (block 606). For example, the device may perform this extraction as described above. As also shown in FIG. 6, process 600 may include assigning weights to the DCH signal, the fundamental signal, and the second harmonic signal to obtain weighted signals of nonlinear frequencies (block 608). For example, the device may assign weights as described above. Finally, as shown in FIG. 6, process 600 may include generating a final image based on the weighted signals of nonlinear frequencies (block 610). For example, the device may generate the final image based on the weighted signals as described above.

Process 600 may include additional implementations, such as any single implementation or any combination of implementations described below and/or in connection with one or more other processes described elsewhere herein. In a first implementation, the DCH signal corresponds to a low-frequency component centered around 0 Hz. In a second implementation, alone or in combination with the first implementation, the weights are assigned based on at least one of imaging depth or clinical application mode. In a third implementation, alone or in combination with the first and second implementations, the DCH signal is assigned a higher weight to improve image penetration, reduce clutter levels, and enhance border visibility in the final image. In a fourth implementation, alone or in combination with one or more of the first through third implementations, the second harmonic signal is assigned a higher weight to improve image clarity and resolution of the final image. In a fifth implementation, alone or in combination with one or more of the first through fourth implementations, assigning respective weights is based on a predefined clinical imaging scenario selected from the group consisting of fetal skull imaging, trans-cranial imaging, needle visualization, and deep tissue imaging. In a sixth implementation, alone or in combination with one or more of the first through fifth implementations, extracting the DCH signal and the second harmonic signal may include summing the received echo signals, and extracting the fundamental signal may include subtracting the received echo signals. In a seventh implementation, alone or in combination with one or more of the first through sixth implementations, the DCH signal corresponds to a low-frequency component centered around 0 Hz. In an eighth implementation, alone or in combination with one or more of the first through seventh implementations, extracting the DCH signal and the second harmonic signal may include summing the received echo signals, and extracting the fundamental signal may include subtracting the received echo signals.

Although FIG. 6 shows example blocks of process 600, in some implementations, process 600 may include additional blocks, fewer blocks, different blocks, or differently arranged blocks than those depicted in FIG. 6. Additionally or alternatively, two or more of the blocks of process 600 may be performed in parallel.

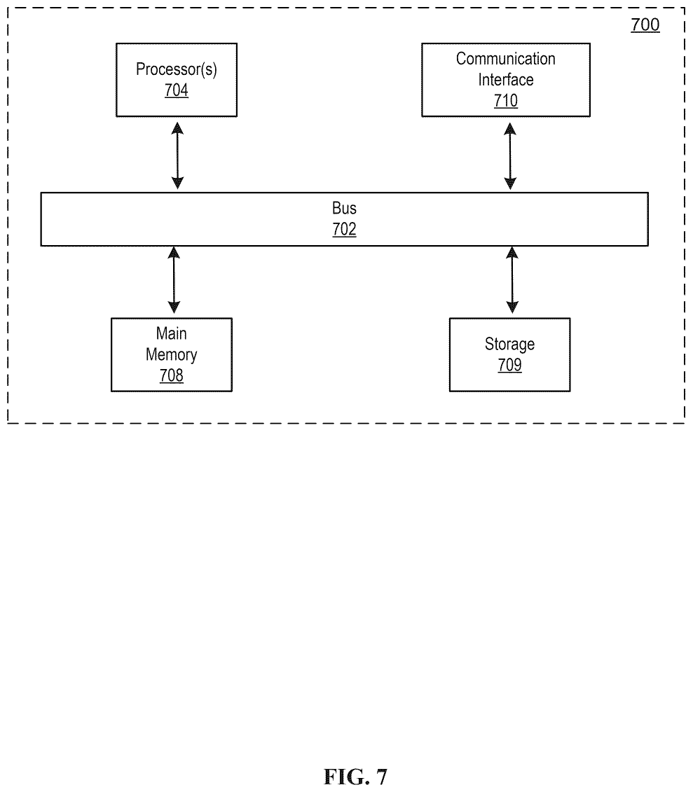

FIG. 7 illustrates an example computing device in which any of the embodiments described herein may be implemented. The computing device 700 may be used to implement one or more components of the systems and the methods shown in FIGS. 1-6 The computing device 700 may comprise a bus 702 or other communication mechanism for communicating information and one or more hardware processors 704 coupled with bus 702 for processing information. Hardware processor(s) 704 may be, for example, one or more general purpose microprocessors.

The computing device 700 may also include a main memory 708, such as a random-access memory (RAM), cache and/or other dynamic storage devices, coupled to bus 702 for storing information and instructions to be executed by processor(s) 704. Main memory 708 also may be used for storing temporary variables or other intermediate information during execution of instructions to be executed by processor(s) 704. Such instructions, when stored in storage media accessible to processor(s) 704, may render computing device 700 into a special-purpose machine that is customized to perform the operations specified in the instructions. Main memory 708 may include non-volatile media and/or volatile media. Non-volatile media may include, for example, optical or magnetic disks. Volatile media may include dynamic memory. Common forms of media may include, for example, a floppy disk, a flexible disk, hard disk, solid state drive, magnetic tape, or any other magnetic data storage medium, a CD-ROM, any other optical data storage medium, any physical medium with patterns of holes, a RAM, a DRAM, a PROM, and EPROM, a FLASH-EPROM, NVRAM, any other memory chip or cartridge, or networked versions of the same.

The computing device 700 may implement the techniques described herein using customized hard-wired logic, one or more ASICs or FPGAs, firmware and/or program logic which in combination with the computing device may cause or program computing device 700 to be a special-purpose machine. According to one embodiment, the techniques herein are performed by computing device 700 in response to processor(s) 704 executing one or more sequences of one or more instructions contained in main memory 708. Such instructions may be read into main memory 708 from another storage medium, such as storage device 709. Execution of the sequences of instructions contained in main memory 708 may cause processor(s) 704 to perform the process steps described herein. For example, the processes/methods disclosed herein may be implemented by computer program instructions stored in main memory 708. When these instructions are executed by processor(s) 704, they may perform the steps as shown in corresponding figures and described above. In alternative embodiments, hard-wired circuitry may be used in place of or in combination with software instructions.

The computing device 700 also includes a communication interface 710 coupled to bus 702. Communication interface 710 may provide a two-way data communication coupling to one or more network links that are connected to one or more networks. As another example, communication interface 710 may be a local area network (LAN) card to provide a data communication connection to a compatible LAN (or WAN component to communicate with a WAN). Wireless links may also be implemented.

The performance of certain of the operations may be distributed among the processors, not only residing within a single machine, but deployed across a number of machines. In some example embodiments, the processors or processor-implemented engines may be located in a single geographic location (e.g., within a home environment, an office environment, or a server farm). In other example embodiments, the processors or processor-implemented engines may be distributed across a number of geographic locations.

Each of the processes, methods, and algorithms described in the preceding sections may be embodied in, and fully or partially automated by, code modules executed by one or more computer systems or computer processors comprising computer hardware. The processes and algorithms may be implemented partially or wholly in application-specific circuitry.

When the functions disclosed herein are implemented in the form of software functional units and sold or used as independent products, they can be stored in a processor executable non-volatile computer readable storage medium. Particular technical solutions disclosed herein (in whole or in part) or aspects that contribute to current technologies may be embodied in the form of a software product. The software product may be stored in a storage medium, comprising a number of instructions to cause a computing device (which may be a personal computer, a server, a network device, and the like) to execute all or some steps of the methods of the embodiments of the present application. The storage medium may comprise a flash drive, a portable hard drive, ROM, RAM, a magnetic disk, an optical disc, another medium operable to store program code, or any combination thereof.

Particular embodiments further provide a system comprising a processor and a non-transitory computer-readable storage medium storing instructions executable by the processor to cause the system to perform operations corresponding to steps in any method of the embodiments disclosed above. Particular embodiments further provide a non-transitory computer-readable storage medium configured with instructions executable by one or more processors to cause the one or more processors to perform operations corresponding to steps in any method of the embodiments disclosed above.

Embodiments disclosed herein may be implemented through a cloud platform, a server or a server group (hereinafter collectively the “service system”) that interacts with a client. The client may be a terminal device, or a client registered by a user at a platform, wherein the terminal device may be a mobile terminal, a personal computer (PC), and any device that may be installed with a platform application program.

The various features and processes described above may be used independently of one another or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The exemplary systems and components described herein may be configured differently than described. For example, elements may be added to, removed from, or rearranged compared to the disclosed example embodiments.

The various operations of exemplary methods described herein may be performed, at least partially, by an algorithm. The algorithm may be compromised in program codes or instructions stored in a memory (e.g., a non-transitory computer-readable storage medium described above). Such algorithm may comprise a machine learning algorithm. In some embodiments, a machine learning algorithm may not explicitly program computers to perform a function but can learn from training data to make a prediction model that performs the function.

The various operations of exemplary methods described herein may be performed, at least partially, by one or more processors that are temporarily configured (e.g., by software) or permanently configured to perform the relevant operations. Whether temporarily or permanently configured, such processors may constitute processor-implemented engines that operate to perform one or more operations or functions described herein.

Similarly, the methods described herein may be at least partially processor-implemented, with a particular processor or processors being an example of hardware. For example, at least some of the operations of a method may be performed by one or more processors or processor-implemented engines. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an Application Program Interface (API)).

The performance of certain of the operations may be distributed among the processors, not only residing within a single machine, but deployed across a number of machines. In some example embodiments, the processors or processor-implemented engines may be located in a single geographic location (e.g., within a home environment, an office environment, or a server farm). In other example embodiments, the processors or processor-implemented engines may be distributed across a number of geographic locations.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the subject matter has been described with reference to specific example embodiments, various modifications and changes may be made to these embodiments without departing from the broader scope of embodiments of the present disclosure. Such embodiments of the subject matter may be referred to herein, individually or collectively, by the term “invention” merely for convenience and without intending to voluntarily limit the scope of this application to any single disclosure or concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

As used herein, “or” is inclusive and not exclusive, unless expressly indicated otherwise or indicated otherwise by context. Therefore, herein, “A, B, or C” means “A, B, A and B, A and C, B and C, or A, B, and C,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context. Moreover, plural instances may be provided for resources, operations, or structures described herein as a single instance. Additionally, boundaries between various resources, operations, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

The term “include” or “comprise” is used to indicate the existence of the subsequently declared features, but it does not exclude the addition of other features. Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

1. An ultrasound imaging system, comprising:
a controller configured to transmit a pulse inversion (PI) sequence including a positive ultrasound pulse and a negative ultrasound pulse via an ultrasound probe into biological tissue;
a signal processing circuit configured to:
receive echo signals resulting from the PI sequence;
extract (i) a direct current harmonic (DCH) signal, (ii) a fundamental signal presenting a frequency of the positive ultrasound pulse and the negative ultrasound pulse, and (iii) a second harmonic signal based on the echo signals;
assign weights to the DCH signal, the fundamental signal, and the second harmonic signal to obtain weighted signals of nonlinear frequencies; and
generate a final image based on the weighted signals of nonlinear frequencies.

2. The ultrasound imaging system of claim 1, wherein the DCH signal corresponds to a low frequency component centered around 0 Hz.

3. The ultrasound imaging system of claim 1, wherein the weights assigned to the DCH signal, the fundamental signal, and the second harmonic signal are determined based on at least one of imaging depth or clinical application mode.

4. The ultrasound imaging system of claim 1, wherein the DCH signal is assigned with a higher weight to improve image penetration, reduce clutter levels, and enhance border visibility in the final image.

5. The ultrasound imaging system of claim 1, wherein the second harmonic signal is assigned with a higher weight to improve image clarity and resolution of the final image.

6. The ultrasound imaging system of claim 1, wherein the weights assigned to DCH signal, the fundamental signal, and the second harmonic signal are determined based on a predefined clinical imaging scenario selected from the group consisting of: fetal skull imaging, trans-cranial imaging, needle visualization, and deep tissue imaging.

7. The ultrasound imaging system of claim 1, wherein the signal processing circuit is configured to extract the DCH signal and the second harmonic signal via summation of the received echo signals, and to extract the fundamental signal via subtraction of the received echo signals.

8. The ultrasound imaging system of claim 7, wherein the received echo signals comprise first echo signals of the positive ultrasound pulse and second echo signals of the negative ultrasound pulse.

9. The ultrasound imaging system of claim 1, wherein the DCH signal, the fundamental signal, and the second harmonic signal are derived from nonlinear frequency bands in the echo signals.

10. The ultrasound imaging system of claim 1, wherein to obtain the weighted signals of nonlinear frequencies, the signal processing circuit is further configured to:
generate images respectively based on the DCH signal, the fundamental signal, and the second harmonic signal;
assign the weights to the images to obtain weighted images; and
generate the final image based on the weighted images.

11. A method of ultrasound imaging, comprising:
transmitting a pulse inversion (PI) sequence comprising a positive ultrasound pulse and a negative ultrasound pulse into biological tissue via an ultrasound probe;
receiving echo signals resulting from the PI sequence;
extracting, from the received echo signals, (i) a direct current harmonic (DCH) signal, (ii) a fundamental signal presenting a frequency of the positive ultrasound pulse and the negative ultrasound pulse, and (iii) a second harmonic signal;
assigning weights to the DCH signal, the fundamental signal, and the second harmonic signal to obtain weighted signals of nonlinear frequencies; and
generating a final image based on the weighted signals of nonlinear frequencies.

12. The method of claim 11, wherein the DCH signal corresponds to a low frequency component centered around 0 Hz.

13. The method of claim 11, wherein the weights are assigned based on at least one of imaging depth or clinical application mode.

14. The method of claim 11, wherein the DCH signal is assigned with a higher weight to improve image penetration, reduce clutter levels, and enhance border visibility in the final image.

15. The method of claim 11, wherein the second harmonic signal is assigned with a higher weight to improve image clarity and resolution of the final image.

16. The method of claim 11, wherein the assigning respective weights is based on a predefined clinical imaging scenario selected from the group consisting of: fetal skull imaging, trans-cranial imaging, needle visualization, and deep tissue imaging.

17. The method of claim 11, wherein the extracting the DCH signal and the second harmonic signal comprises summing the received echo signals, and wherein extracting the fundamental signal comprises subtracting the received echo signals.

18. A non-transitory computer-readable medium storing instructions that, when executed by one or more processors, cause the one or more processors to perform operations comprising:
transmitting a pulse inversion (PI) sequence comprising a positive ultrasound pulse and a negative ultrasound pulse into biological tissue via an ultrasound probe;
receiving echo signals resulting from the PI sequence;
extracting, from the received echo signals, (i) a direct current harmonic (DCH) signal, (ii) a fundamental signal presenting a frequency of the positive ultrasound pulse and the negative ultrasound pulse, and (iii) a second harmonic signal;
assigning weights to the DCH signal, the fundamental signal, and the second harmonic signal to obtain weighted signals of nonlinear frequencies; and
generating a final image based on the weighted signals of nonlinear frequencies.

19. The non-transitory computer-readable medium of claim 18, wherein the DCH signal corresponds to a low frequency component centered around 0 Hz.

20. The non-transitory computer-readable medium of claim 18, wherein the extracting the DCH signal and the second harmonic signal comprises summing the received echo signals, and wherein extracting the fundamental signal comprises subtracting the received echo signals.
